(19) United States
(12) Patent Application Publication
(10) Pub. No.: US 2025/0288421 A1
(43) Pub. Date: Sep. 18, 2025

(54) SLEEVE FOR A PROSTHETIC IMPLANT

(71) Applicant: LIMACORPORATE S.P.A., San Daniele Del Friuli (IT)

(72) Inventors: Marco ZUCCHET, Pordenone (IT); Christoph FIEDLER, Diekhof (DE); Michele PRESSACCO, Martignacco (IT)

(21) Appl. No.: 18/875,889

(22) PCT Filed: Jul. 5, 2023
(86) PCT No.: PCT/IT2023/050159

§ 371 Date: Dec. 17, 2024

(30) Foreign Application Priority Data
Jul. 6, 2022 (IT) 102022000014317

(51) Int. Cl. A61F 2/30; A61F 2/36

(52) U.S. Cl. CPC A61F 2/30734; A61F 2/30767; A61F 2/3094; A61F 2/3609; A61F 2/3662; A61F 2002/30235; A61F 2002/30317; A61F 2002/30331; A61F 2002/30616; A61F 2002/30738; A61F 2002/3092; A61F 2002/30985; A61F 2002/3625

(57) ABSTRACT

Sleeve for a prosthetic implant implantable in bone, for example a femur, and related prosthetic assembly including at least a stem, intermediate neck component and sleeve.

### FIELD OF THE INVENTION

The present invention concerns a sleeve for a prosthetic implant known to those skilled in the art with the English term “sleeve”, which is used in surgical procedures for the revision of prosthetic implants, for example femoral, if there is a significant loss of bone in the metaphyseal zone and it is desired to ensure the proximal stability of the prosthetic implant.

### BACKGROUND OF THE INVENTION

It is known that due to degenerative, congenital, or traumatic diseases, for example primary osteoarthritis, secondary osteoarthritis, due to trauma or caused by infections, rheumatoid arthritis, inflammatory arthritis, osteonecrosis, or bone tumours, or other similar problems, it is necessary to perform joint arthroplasty operations which consist of installing a prosthetic implant, or prosthesis, capable of reproducing, overall, a movement analogous to that of a healthy joint.

In the event of revision of the prosthetic implant, it is known to use a sleeve that acts as a filler to fill the gap generated by the primary implant that has failed.

In the specific case of the hip joint, the femoral prosthesis comprises a rod adapted to be inserted into the medullary canal of the femur, a substantially hemispherical head adapted to be inserted into a corresponding acetabular seat of the hip, and an intermediate component known to those skilled in the art with the term “neck” to which the rod is connected on one side and the head on the opposite side.

In revision surgical procedures, the sleeve is inserted in a special seat made with a surgical cutter in the proximal part of the femur and is configured to accommodate the part of the neck connected to the rod to exert a supporting action and correct positioning of the prosthesis. In other words, the sleeve serves to fill the bone loss and at the same time provide a new proximal support so as to unload the rod, preventing the entire load from being unloaded distally and thereby stressing the modular coupling cone.

Sleeves known in the art comprise a metal body having an axial through cavity and an external wall extending about a longitudinal axis. The external wall overall has a cylindrical, truncated cone shape or a combination thereof and its profile is obtained by the rotation of one or more generatrices that are straight lines or broken lines.

A disadvantage of the sleeves known in the art is that the external wall has a profile that is not anatomical or is not capable of following the profile of the femur so that such sleeves can cause an incorrect joint load distribution with a concentration of stress in the contact areas and a reabsorption in the areas that are not loaded.

A further disadvantage of the known sleeves is that their shape may require an excessive removal of bone which may be excessively weakened, or they have a tapered shape that fails to ensure sufficient support to the prosthesis.

U.S. Pat. No. 5,342,366 A discloses a modular hip prosthesis in which the rod and neck are detachably connected. U.S. Pat. No. 5,342,366 A does not disclose a sleeve nor the application of a sleeve for supporting said modular hip prosthesis.

WO 01/43650 A1 discloses a surgical instrument for positioning a femoral rod in a femur, in which the femoral rod, including the neck, is monolithic. WO 01/43650 A1 does not disclose a sleeve nor the application of a sleeve for supporting the femoral rod.

There is therefore the need to perfect a sleeve for a prosthetic implant that can overcome at least one of the disadvantages of the state of the art.

In particular, a purpose of the present invention, which coincides with the technical problem to be solved, is to make a sleeve for a prosthetic implant having an anatomical profile.

A further purpose of the present invention is to make a sleeve which is minimally invasive, i.e., having a shape, conjugated to the corresponding bone site of installation, which satisfies the requirement of minimum removal of bone material possible to ensure the seal of the prosthesis.

A further purpose of the present invention is to provide a prosthetic assembly, formed by a prosthetic implant and a sleeve, which is modular in terms of component sizes, and which is easily assembled and disassembled by the surgeon.

The Applicant has devised, tested and embodied the present invention to overcome the shortcomings of the state of the art and to obtain these and other purposes and advantages.

### SUMMARY OF THE INVENTION

The present invention is set forth and characterized in the independent claim. The dependent claims describe other characteristics of the present invention or variants to the main inventive idea.

In accordance with the above purposes and to resolve the technical problem disclosed above in a new and original way, also achieving considerable advantages compared to the state of the prior art, a sleeve according to the present invention, for a prosthetic implant able to be implanted in a bone, for example in a femur, comprises an external wall defined by a first portion and a second portion, and which has, on said first portion, a first external surface obtained by the rotation of a first generatrix about a first longitudinal axis, and on said second portion, a second external surface obtained by the rotation of a second generatrix about a second longitudinal axis.

In accordance with one aspect of the present invention, said longitudinal axes are reciprocally inclined by a given angle of inclination.

In accordance with one aspect of the present invention, at least said second external surface is at least partially concave toward the inside of said sleeve.

In accordance with another aspect of the present invention, the first generatrix consists of a set of polynomials connected to each other. The second generatrix is an arc of circumference, parabola, ellipse, hyperbolae, or a curve consisting of a set of polynomials connected to each other.

In accordance with another aspect of the present invention, said first generatrix is a broken line, and said second generatrix is concave toward the inside of the sleeve, preferably continuous.

In accordance with another aspect of the present invention, the extension of said first and second external surfaces is variable and depends on the extension of said first and second portions, respectively.

In accordance with another aspect of the present invention, the ratio between the extension of said first and second external surfaces can be comprised between 0 and 5, preferably between 0.20 and 3.

In accordance with another aspect of the present invention, the external surface of said external wall may be constituted, in addition to said first and second external surfaces, also by a third external surface obtained by the prolongation, along said first longitudinal axis, of the profiles of said second portion.

In accordance with another aspect of the present invention, the rotation of said first and second generatrices generates a first and a second volume which define at least said first and second external surfaces, respectively. Moreover, the external surface of said sleeve consists of a part of the external surface of a final volume constituted by the union of said first and second volumes.

In accordance with another aspect of the present invention, said part is obtained by “cutting” said final volume along two cutting planes, which are axially distanced along said longitudinal axis.

In accordance with another aspect of the present invention, said cutting planes are parallel to each other.

In accordance with a variant embodiment, at least one of said cutting planes has a step-shaped profile.

Consequently, said cutting planes determine a higher lateral portion, at the same level, or lower than the medial portion of the sleeve.

In accordance with another aspect of the present invention, said second portion may be provided with one or more through holes which can have a cylindrical, elliptical or spline section. If present, the through hole can be placed at a first distance from the above first longitudinal axis, comprised between 12 and 24 mm, and at a second distance, comprised between 4 and 15 mm, from a proximal end of the sleeve.

In accordance with another aspect of the present invention, said external wall comprises a solid part and a part with a trabecular structure. Furthermore, both said solid part and said part with a trabecular structure are made in a single body by means of additive printing technology. In accordance with a variant, the entire external wall can have a trabecular structure.

In accordance with another aspect of the present invention, the above sleeve comprises an internal surface able to allow a coupling with an intermediate component of a prosthetic implant which, in turn, is configured to couple with a rod of said prosthetic implant.

In accordance with embodiments of the present invention, a modular prosthetic assembly comprises at least three component modules able to be coupled together, of which:
at least one intramedullary rod and an intermediate neck component able to be associated with said rod and with a prosthetic head, said at least one rod and intermediate neck component being comprised in a prosthetic implant;
a sleeve according to the invention, configured to stably couple to said intermediate neck component.

In accordance with another aspect of the present invention, said rod, said intermediate neck component and said sleeve are distinct and separate component modules able to be connected to each other in a selectively decouplable manner.

In accordance with another aspect of the present invention, said prosthetic assembly also comprises said prosthetic head.

In accordance with another aspect of the present invention, said rod, said intermediate component and said sleeve are provided with modular sizes.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1a is a side view of an assembled prosthetic assembly, formed by a sleeve, according to the present invention, coupled with a neck, in turn coupled with a head and a rod forming a prosthetic implant;

FIG. 1b is a separate component view of the prosthetic assembly of FIG. 1a shown in a disassembled condition;

FIG. 2 is a front axonometric view of the sleeve of FIGS. 1a and 1b;

FIG. 3 is a longitudinal sectional view of the sleeve of FIG. 2;

FIG. 4 is a top view of the sleeve of FIG. 2;

FIGS. 5-6 are embodiment variants of FIG. 3;

FIGS. 7-12 schematically illustrate the volumes defining the external surface of the sleeve in a combined manner;

FIGS. 13-15 schematically illustrate how the sleeve of the present invention is obtained from the combination of the volumes of FIGS. 7-12 sectioned along two cutting planes.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

With reference to FIGS. 1a and 1b, a sleeve 10 according to the present invention is configured to support in use a prosthetic implant 100 inside a portion of bone (not shown).

The prosthetic implant 100 together with the sleeve 10 forms a prosthetic assembly 200 which is modular, both in terms of assembly/disassembly and in terms of component sizes/dimensions.

The prosthetic implant 100 described herein is for example a femoral prosthesis.

The prosthetic implant 100 comprises an intramedullary rod 110, for example able to be inserted into the medullary canal of a femur, and an intermediate component, or neck, 112 to which the rod 110 is associated on one side.

Furthermore, in possible embodiments the prosthetic implant 100 can comprise a substantially hemisphere-shaped head 111 able to be inserted into a corresponding acetabular site of the hip. In this case, the above intermediate component, or neck, 112 is associated with the head 111 on the opposite side with respect to the rod 110.

As clearly shown in FIGS. 1b, the modular prosthetic assembly 200 is formed both by the prosthetic implant 100, which comprises distinct components or modules, i.e., rod 110, neck 112, and possibly head 111, and by the sleeve 10, decouplably connected to each other to create a modular structure.

The sleeve 10 defines a support for the prosthetic implant 100 in the coupling zone between rod 110 and neck 112.

The connection between the components can occur by interlocking possibly with the aid of mechanical locking members of traditional type.

The sleeve 10 of the present invention has a shape which makes it particularly suitable as a bone filler at most up to the internal cortical wall of the femur.

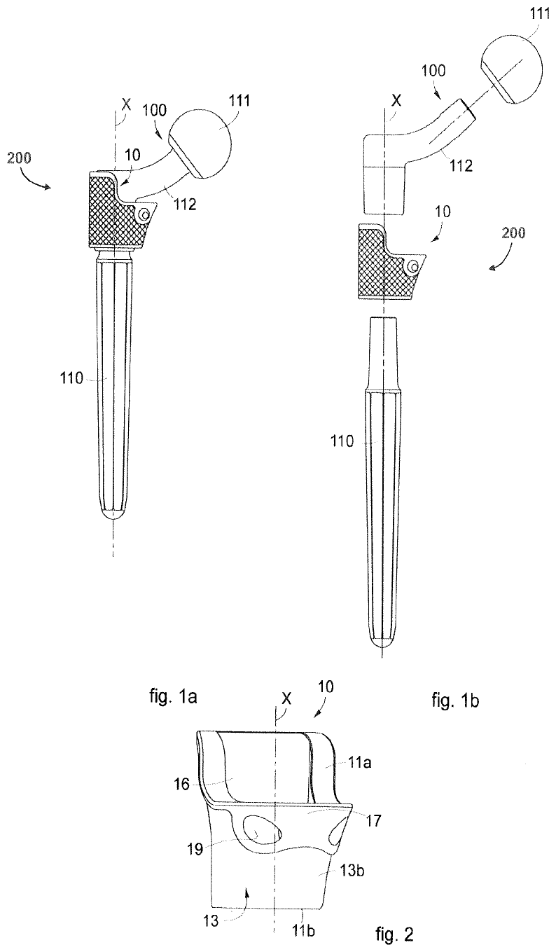

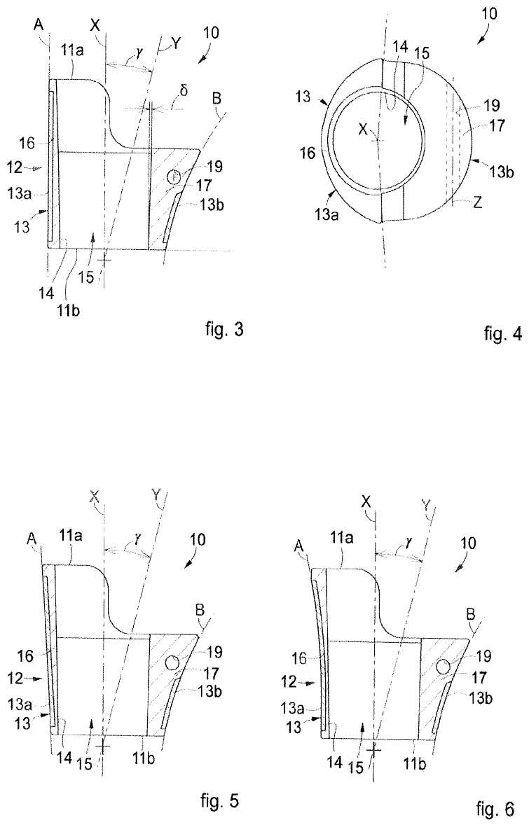

With particular reference to FIGS. 2 and 3, the sleeve 10 extends between a proximal end 11a and a distal end 11b and has an external wall 13 defined by the union of a first portion 16 and a second portion 17.

The external wall 13 extends circumferentially partly around a first longitudinal axis X, in a first external surface 13a, and partly around a second longitudinal axis Y inclined with respect to the latter by an angle of inclination γ, in a second external surface 13b.

The angle of inclination γ can be comprised between 0° and 20°, preferably between 8° and 12°.

The first external surface 13a and the second external surface 13b overall define the external surface of the wall 13, or together with a third external surface 13c as will be explained in more detail below.

Overall, the sleeve 10 has an asymmetric frustoconical shape whereby it is transversely wider at the proximal end 11a with respect to the distal end 11b.

The sleeve 10 also has an internal surface 14, opposite the external surface of the wall 13, which defines a through hole 15, coaxial with the first longitudinal axis X and configured to receive the intermediate component 112 to which the rod 110 is in turn connected.

The hole 15 has a substantially truncated cone longitudinal section, conjugated with the external profile of the intermediate component 112 so as to define a shape coupling or coupling by interference therewith. For example, the hole 15 can have a taper δ which goes from about 1° to about 20°, preferably less than 10°, more preferably between about 2° and 5°.

Consequently, the sleeve 10 can have a progressively reduced thickness, due to the taper δ of the hole 15.

The internal surface 14 is substantially symmetrical with respect to the first longitudinal axis X throughout the extension thereof.

In contrast, the external wall 13 is for a part symmetric with respect to the first longitudinal axis X (first external surface 13a) and for a part symmetric with respect to the second longitudinal axis Y (second external surface 13b).

The first external surface 13a, which externally defines the first portion 16, is obtained by rotating a first generatrix A about the first longitudinal axis X.

The second external surface 13b, which externally defines the second portion 17, is obtained by rotating a second generatrix B about the second longitudinal axis Y.

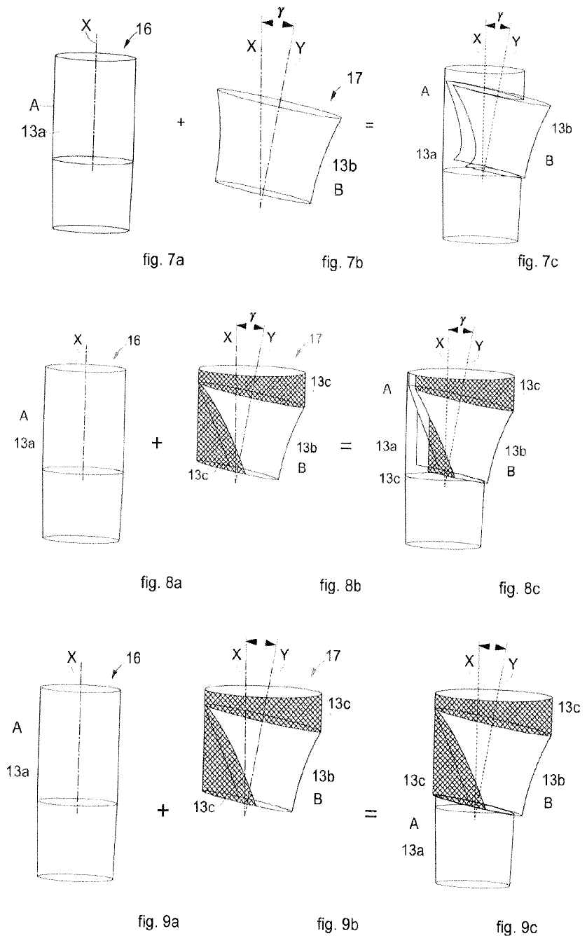

With particular reference to FIGS. 7-12, the volumes defining the external surfaces 13a, 13b, 13c are illustrated which, suitably combined, generate a final volume which determines the profile of the external surface of the wall 13. FIGS. 7a-12a and 7b-12b show the elementary volumes of the first and the second portions 16, 17 while FIGS. 7c-12c show the combination of said elementary volumes.

As can be appreciated, the extension of the first and second external surfaces 13a and 13b is variable and depends on the extension of the portions 16 and 17, respectively.

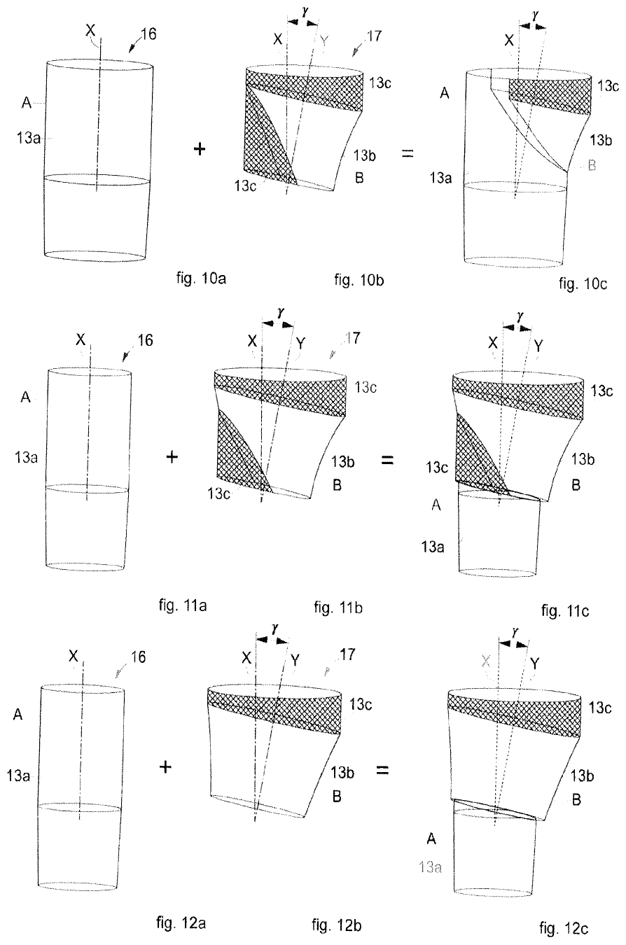

In particular, the ratio between the extension of the first and second external surfaces 13a and 13b is variable, as shown, for example, in FIGS. 9a-9c and 10a-10c, where respectively in FIGS. 9a-9c the second external surface 13b is larger than the first external surface 13a while in FIGS. 10a-10c the second external surface 13b is smaller than the first external surface 13a.

According to embodiments, the ratio between the extension of the first and second external surfaces 13a, 13b can be comprised between 0 and 5, preferably between 0.20 and 3.

With particular reference to FIGS. 8c-12c, the external surface of the wall 13 can be constituted, in addition to the external surfaces 13a and 13b, also by a third external surface 13c obtained by the prolongation, along the first longitudinal axis X, of the profiles of the second portion 17 in one or both axial directions, FIGS. 8b-12b.

The external surface of the sleeve 10 can consist of a part of the external surface of the final volume constituted by the union of said elementary volumes.

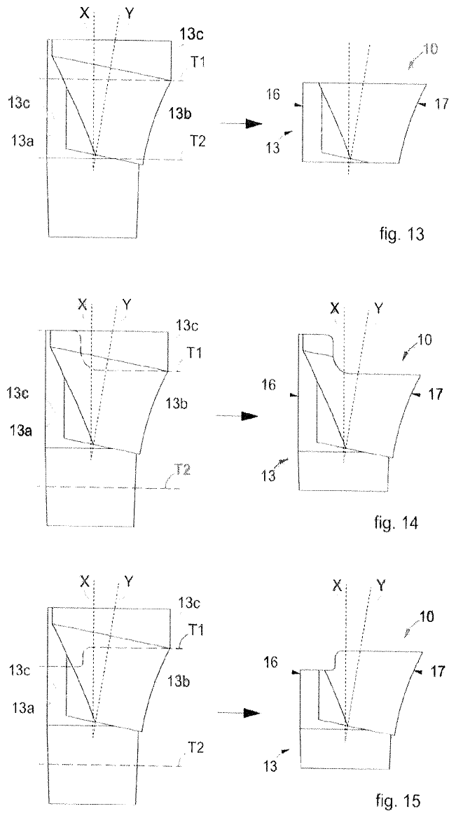

Said part can be obtained by “cutting” the final volume along two cutting planes T1, T2 which are axially distanced along the first longitudinal axis X, FIGS. 13-15.

According to embodiments, the cutting planes T1, T2 can be parallel to each other, FIG. 13.

According to further embodiments, at least one of the cutting planes T1, T2 can have a step-shaped profile, FIGS. 14, 15.

The cutting of the surface can result in a lateral portion, at the same level (FIG. 13), higher (FIG. 14) or lower (FIG. 15) than the medial portion of the sleeve 10.

In other words, the surface 13 can be cut, following a multitude of profiles, so as to generate a subset of new surfaces.

In accordance with a further aspect of the present invention, the first generatrix is a straight line or spline, i.e., a curve consisting of a set of polynomials connected to each other. The second generatrix is an arc of circumference, parabola, ellipse, hyperbolae, or a spline curve.

The first generatrix A can be a broken line or a straight line which is inclined with respect to the first longitudinal axis X, for example by an angle comprised between about 1° and about 15° (FIG. 5).

In further embodiments, the first generatrix A has a concave profile (FIG. 6) towards the inside.

The second generatrix B is a preferably continuous concave curve whereby the second external surface 13b has a profile concave toward the inside, i.e., toward the first longitudinal axis X.

The second generatrix B can be a branch of circumference, parabola, ellipse, hyperbolae or a spline curve, i.e., a curve consisting of a set of polynomials connected to each other. If the second generatrix B is an arc of circumference, the circumference can have a radius comprised between 20 and 200 mm.

The second portion 17 is provided with a through hole 19 which extends along a transversal axis Z (FIG. 4) and is configured to receive a metal cable during a reconstruction operation of the proximal portion of the femur in the event of fractures.

At least one part of the external wall 13 can have a porous, or trabecular, structure with the purpose of promoting the osseointegration of the bone in contact with the sleeve 10.

The body of the sleeve 10 is made in a single piece by means of additive printing technology.

According to embodiments, the sleeve 10 according to the present invention is suitable to be inserted both in a cavity obtained with the use of a traditional milling device and, more favourably, obtained with the use of the guided milling device for prosthetic surgery described in the international application WO2021/186487 A1 of the same Applicant.

It is clear that modifications and/or additions of parts may be made to the sleeve 10 for a prosthetic implant 100 described heretofore, without departing from the scope of the present invention, as defined by the claims.

It is also clear that, although the present invention has been described with reference to some specific examples, a person of skill in the art shall certainly be able to achieve other equivalent forms of a sleeve 10 for a prosthetic implant 100, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. Sleeve (10) for a prosthetic implant (100) able to be implanted in a bone, wherein said sleeve (10) comprises an external wall (13) defined by a first portion (16) and a second portion (17), and which has, on said first portion (16), a first external surface (13a) obtained by the rotation of a first generatrix (A) about a first longitudinal axis (X), and, on said second portion (17), a second external surface (13b) obtained by the rotation of a second generatrix (B) about a second longitudinal axis (Y), wherein said longitudinal axes (X, Y) are reciprocally inclined by an angle of inclination (γ), and in that at least said second external surface (13b) is at least partly concave toward the inside of said sleeve (10).

2. Sleeve (10) as in claim 1, wherein that said first generatrix (A) consists of a set of polynomials connected to each other, and said second generatrix (B) is an arc of circumference, parabola, ellipse, hyperbolae, or a curve consisting of a set of polynomials connected to each other.

3. Sleeve (10) as in claim 1, wherein said first generatrix (A) is a broken line or a straight line parallel to said first longitudinal axis (X), and said second generatrix (B) is concave toward the inside of the sleeve (10).

4. Sleeve (10) as in claim 1, wherein the extension of said first and second external surfaces (13a, 13b) is variable, and it depends on the extension of said first and second portions (16, 17), respectively.

5. Sleeve (10) as in claim 1, wherein the ratio between the extension of said first and second external surfaces (13a, 13b) is comprised between 0 and 5, preferably between 0.20 and 3.

6. Sleeve (10) as in claim 1, wherein the external surface of said external wall (13) also consists of, in addition to said first and second external surfaces (13a, 13b), a third external surface (13c) obtained by the prolongation, along said first longitudinal axis (X), of the profiles of said second portion (17).

7. Sleeve (10) as in claim 1, wherein the rotation of said first and second generatrices (A, B) generates a first and a second volume which define at least said first and second external surfaces (13a, 13b) respectively, wherein the external surface of said sleeve (10) consists of a part of the external surface of a final volume constituted by the union of said first and second volumes.

8. Sleeve (10) as in claim 7, wherein said part is obtained by “cutting” said final volume along two cutting planes (T1, T2) which are axially distanced along said first longitudinal axis (X).

9. Sleeve (10) as in claim 8, wherein said cutting planes (T1, T2) are parallel to each other.

10. Sleeve (10) as in claim 8, wherein at least one of said cutting planes (T1, T2) has a step-shaped profile.

11. Sleeve (10) as in claim 1, wherein said second portion (17) is provided with one or more through holes (19) which can have a cylindrical, elliptical or spline section.

12. Sleeve (10) as in claim 1, wherein said external wall (13) comprises a solid part and a part with a trabecular structure, and in that both said solid part and also said part with a trabecular structure are made in single body by means of additive printing technology.

13. Sleeve (10) as in claim 1, wherein the entire external wall (13) has a trabecular structure and is made in a single body by means of additive printing technology.

14. Sleeve (10) as in claim 1, wherein it comprises an internal surface (14) able to allow a coupling with an intermediate component (112) of a prosthetic implant (100) which, in turn, is configured to couple with a rod (110) of said prosthetic implant (100).

15. Modular prosthetic assembly (200) comprising at least three component modules (10, 110, 112) able to be coupled together, of which:
at least one intramedullary rod (110) and an intermediate neck component (112) able to be associated with said rod (110) and with a prosthetic head (111), said at least one rod (110) and intermediate neck component (112) being comprised in a prosthetic implant (100);
a sleeve (10), as in claim 1, configured to stably couple to said intermediate neck component (112).

16. Modular prosthetic assembly (200) as in claim 15, wherein at least said rod (110), said intermediate neck component (112) and said sleeve (10) are distinct and separate component modules able to be connected to each other in a selectively decouplable manner.

17. Modular prosthetic assembly (200) as in claim 15, wherein it also comprises said prosthetic head (111).

18. Modular prosthetic assembly (200) as in claim 15, wherein said rod (110), said intermediate component (112) and said sleeve (10) are provided with modular sizes.
